(19) United States
(12) Patent Application Publication
(10) Pub. No.: US 2026/0273181 A1
(43) Pub. Date: Sep. 17, 2026

(54) INFUSION PUMP DISPOSABLE CHARACTERIZATION AND COMPATIBILITY

(71) Applicant: CareFusion 303, Inc., San Diego, CA (US)

(72) Inventors: Bailey Marie FLOREK, Morristown, NJ (US); Michael K. WORKMAN, Carlsbad, CA (US)

(21) Appl. No.: 19/080,563

(22) Filed: Mar. 14, 2025

(51) Int. Cl. A61M 5/172; G16H 20/17

(52) U.S. Cl. CPC A61M 5/172; G16H 20/17

(57) ABSTRACT

Certain aspects of the disclosure provide systems and methods for detecting compatibility between infusion systems and infusion administration sets, in particular between infusion administration sets and infusion fluids. Some methods include determining information characterizing the infusion administration set and detecting at the infusion device, compatibility between the infusion administration set and the fluid based on the information characterizing the infusion administration set.

### BACKGROUND

### Field

Aspects of the present disclosure relate to detection of compatibility of infusion system, for example, compatibility between infusion administration sets and infusion orders.

### Introduction

Infusion systems are a common medical device for administration of therapeutic fluids to a subject, such as intravenously. Previously, fluid containers were elevated to utilize gravity to drop the fluid through a line to the subject.

Now, a pump is used to control flow of the therapeutic fluid to the subject in an accurate and consistent manner. The therapeutic fluid container may be connected to the subject through an assembly of tubing, valves, fittings, connectors, chambers, and needles. This assembly may be referred to as an infusion administration set. An infusion administration set is typically disposable to reduce infection and contamination risk.

A typical infusion system a therapeutic fluid from a fluid container through an infusion administration set. A pumping segment or component of the infusion administration set is located inside pumping module the infusion pump. An infusion pump controller controls the pumping module to pump fluid from the fluid container through the infusion administration set.

### SUMMARY

Certain aspects provide a method comprising: receiving, at an infusion device, an identifier for a fluid administration set; determining, by the infusion device, information characterizing the fluid administration set based on the identifier; receiving, at the infusion device, an identifier for a fluid to be administered via the fluid administration set; detecting, at the infusion device and based on the information characterizing the fluid administration set and the identifier for the fluid, compatibility between the fluid administration set and the fluid; and one of: in response to detecting the fluid administration set is incompatible with the fluid, outputting, via an electronic display associated with the infusion device, an indication that the fluid administration set is incompatible with the fluid; or in response to detecting the fluid administration set is compatible with the fluid, activating a first control element configured to initiate administration of the fluid by the infusion device.

Other aspects provide processing systems configured to perform the aforementioned methods as well as those described herein; non-transitory, computer-readable media comprising instructions that, when executed by a processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more aspects.

### DESCRIPTION OF THE DRAWINGS

The appended figures depict certain aspects and are therefore not to be considered limiting of the scope of this disclosure.

FIG. 1 depicts an example compatibility system for detecting infusion administration set compatibility with an infusion order.

FIG. 2 depicts an example workflow for detecting infusion administration set compatibility with an infusion order.

FIG. 3 depicts an example workflow for generating a compatibility table for infusion administration sets.

FIG. 4 depicts an example compatibility table for infusion administration sets.

FIG. 5 depicts an example infusion system.

FIG. 6 depicts an example syringe infusion system.

FIG. 7 depicts an example method for detecting infusion administration compatibility with an infusion order.

FIG. 8 depicts an example computing device with which aspects of the present disclosure can be performed.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for detecting of compatibility of an infusion system, for example, compatibility between infusion administration sets and infusion orders.

Infusion administration sets may comprise an assembly of components configured to facilitate administration of a therapeutic fluid to a subject through an infusion pump, for example, an intravenous (IV) set connecting the subject to a therapeutic fluid container and pump.

During set up of the infusion system, components of the infusion administration set may be coupled together, and to the subject, through a patient IV needle, e.g., disposed intravenously in the subject. The infusion administration set may be further coupled to the fluid container containing the therapeutic fluid. An infusion pump is further coupled and configured to pump the therapeutic fluid from the fluid container to the subject through the infusion administration set.

Generally, certain types of infusion administration sets are compatible with certain types of infusion pumps. For example, compatibility may be based on manufacturer, connection types, pumping mechanism, operational parameters, and the like of the infusion pump.

Different types of infusion administration sets may be configured for use with different types of infusion systems. For example, a first infusion administration set may be configured for use with a syringe pump, while a second infusion administration set may be configured for use with a large-volume-pump. In some cases, the type of infusion administration set may depend on the type of pumping mechanism used by the infusion system.

Further, different types of infusion administration sets may be configured for use with different operational parameters, for example, flow rate, total volume for infusion, total time of infusion, and the like. This may be due to the size of the infusion administration set, such as the diameter of tubing, connectors, valves, and the like of the infusion administration set. In some cases, it may be due to the materials of the infusion administration set.

An incompatible infusion administration set used with an infusion pump may result in an inaccurate or incomplete infusion, e.g., a failure of administration of the therapeutic fluid. The subject may not receive the needed therapy, receive an incorrect dosage, the therapeutic fluid may leak or spill, the infusion system may be exposed to contaminants, etc.

During set up, a clinician may load any infusion administration set compatible with the infusion pump. In some cases, an infusion pump may be compatibility with a number of infusion administration sets. However, every infusion administration set compatible with the infusion pump may not be compatible with the particular infusion order.

For example, a large volume infusion pump (LVP) may be configured to infuse both saline and blood products, but every infusion administration set compatible with the LVP may not be compatible with saline or blood products. Blood products may user a larger diameter (e.g., large-bore) tubing, particular filter(s), locks, connectors, etc., because of the characteristics of blood products for infusion. For example, blood products may be more viscous compared to other fluids, like saline, or use specialized filters for contaminants or clots in the blood product. Thus, a small bore (e.g., small diameter) tubing may not be compatible with a blood product infusion, even though the small bore tubing may be compatible with the LVP.

Other fluids for infusion may similarly not be compatible with certain types of infusion administration sets, for example, due to viscosity of the fluid, interaction between the fluid and the material of the infusion administration set, size of the components (e.g., filters), vents, and other characteristics of the fluid and/or the infusion administration set.

Additionally, other factors, such as the operational parameters for the infusion order, such as flow rate, total volume, or total time of use, may affect compatibility of the infusion administration set. Example additional factors may include environmental conditions of the infusion system, age of the infusion administration system, or patient or care center characteristics.

Aspects described herein provide a technical solution through infusion system detection of compatibility of a loaded infusion administration set with the infusion order, for example, to reduce use of incompatible devices and prevent inaccurate therapeutic administration. An infusion system may detect incompatibility of the infusion administration set and warn of, or prevent use of, the incompatible infusion administration set. In particular, the infusion system may detect incompatibility between the infusion administration set and the infusion order, including the therapeutic fluid. Further, in some aspects, the infusion system may detect incompatibility between operational parameters, environmental conditions, patient characteristics, and the like, for the infusion order. Thus, usage of incompatible infusion administration sets may be reduced. Clinician loading of the infusion system may be improved through notification or warning of the incompatible infusion administration set. Accuracy and performance of infusion systems may be improved through usage of compatible infusion administration sets. Patient safety may also be improved through the improved accuracy and performance of the infusion pump, and reduction of risk of leak or contamination.

Additionally, the compatibility detection system and methods described herein may be utilized in both manual and auto programmed workflows (e.g., manual, closed-loop, or semi-closed loop operation of an infusion system). For example, in manual workflows, a clinician may be notified, an incompatibility between the infusion set and the infusion pump may automatically documented in an electronic medical record, or the infusion pump may prevent commencement of an infusion order due to a detected incompatibility. Similarly, in a semi-closed loop workflow, an infusion system may notify aa clinician of the incompatibility between the infusion set and the infusion pump, and automatically document the detected incompatibility. Further, the infusion system may prevent commencement of the infusion order and/or require a clinician to override the detected incompatibility to use the incompatible infusion administration set with the infusion pump. In a closed-loop system, the infusion system may prevent commencement of an infusion order where an incompatible infusion administration set is detected and document the detection of the incompatibility.

### Example Compatibility Detection System

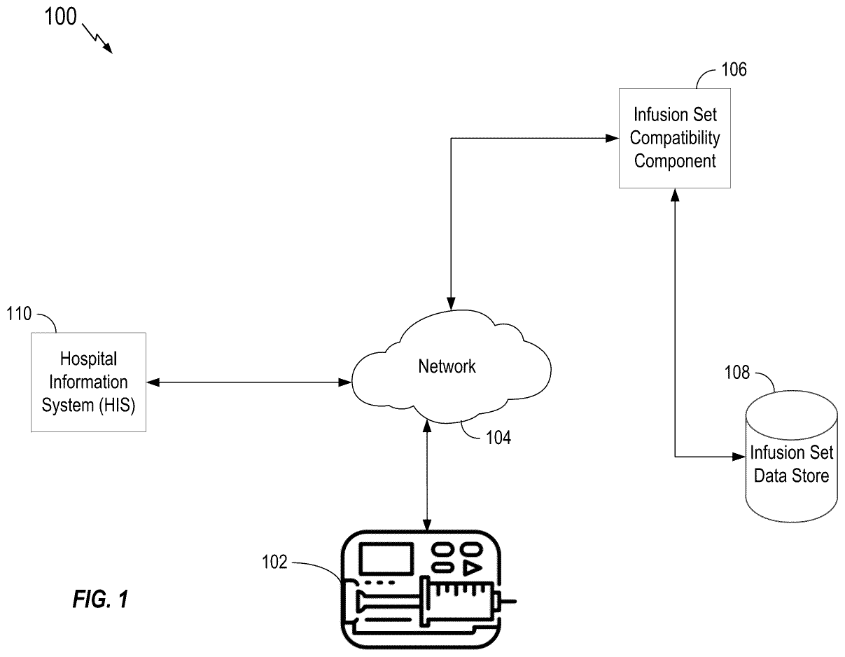

FIG. 1 depicts an example system 100 for monitoring compatibility of infusion administration sets and fluids for administration by infusion systems. In the depicted example, system 100 includes one infusion system 102, although additional infusion systems may be included.

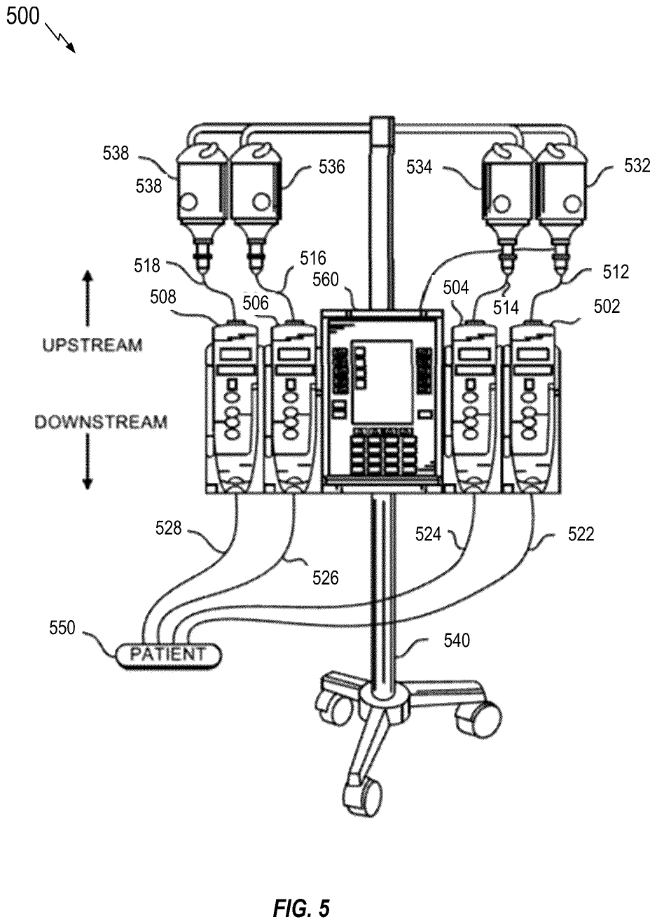

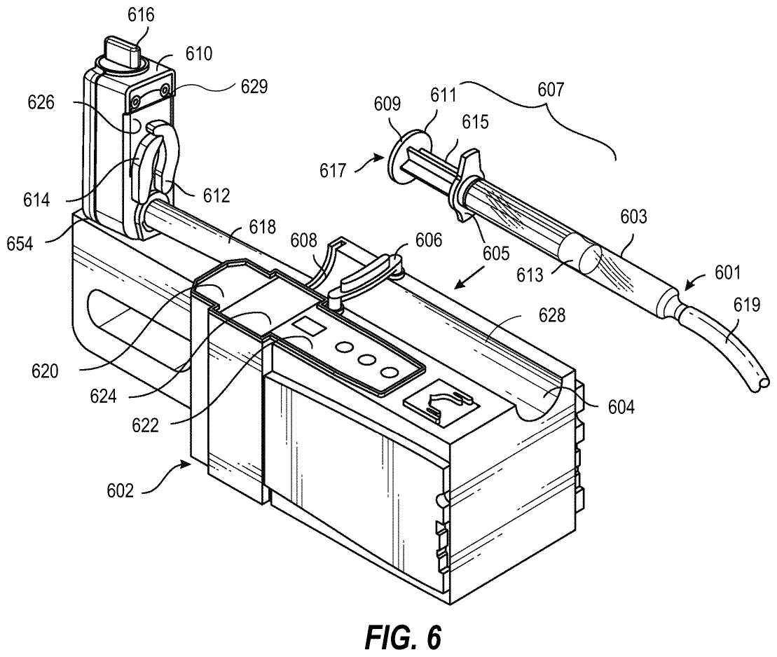

Infusion system 102 is an electromechanical device for infusing therapeutic fluids into a patient in a controlled and precise manner, e.g., according to an infusion order. Exemplary infusion systems may include a large volume infusion pump, such as depicted in FIG. 5, or a syringe pump, such as depicted in FIG. 6. Infusion system 102 comprises an infusion system controller (not depicted) configured to control operations of infusion system 102. In general operation, therapeutic fluid is loaded into an infusion pump, for example, in a syringe, bottle, bag, or other storage container, and the infusion system is configured to pump, or infuse, the therapeutic fluid according to operational parameters of the pump, including, for example, flow rate, volume, and/or time to administer a specific dose of treatment.

The infusion pump is coupled to the subject through one or more components, collectively, an infusion administration set. The infusion administration set may include tubing, filters, valves, connectors, luers, locks, needles, syringes, chambers, and the like, to enable the therapeutic fluid to be administered to the patient, for example, through an intravenous needle. The infusion administration set may comprise a set of components configured to couple together for administration of the therapeutic fluid. An example infusion administration set may include a bag spike that is configured to connect to a fluid container. The bag spike may be connected through tubing to a pumping segment that is then connected through additional tubing to a fitting. A fitting may be a needleless Luer connector suitable for connection to a patient infusion device, such as an intravenous needle. The pumping segment may be a section of tubing suitable for peristaltic manipulation to cause fluid to flow through the tubing and includes two alignment fittings that facilitate proper placement of the pumping segment in the pumping module. The infusion administration set may further include a clamp that can be closed to stop flow through the tubing. The infusion administration set may also include a Y-coupling, a needless access portion, or other components.

Different infusion pumps may be configured for use with different types of infusion administration sets. For example, a syringe-type infusion administration set may be configured for use with a syringe pump. Additionally, different types of infusion administration sets contain different sizes of devices (e.g., of individual pieces of the infusion set), are made with different materials or coatings, or may contain different devices or combinations of devices in a single set. The different types of infusion administration sets may be configured for use in different scenarios based on these features. For example, smaller sizes may be configured to use with smaller patients (e.g., neonates or pediatrics), for different locations, lower flow rates, and the like.

In some aspects, an infusion administration set may comprise one or more devices configured to couple together for administration of a therapeutic fluid with the infusion system. In some examples, the components of the infusion administration set may be produced by a manufacturer in a set, as individual components, or one or more subsets, but otherwise configured for use together as a set.

In some examples, an infusion administration set may include one or more disposable devices, configured for one infusion, such as to reduce infection and contamination. Thus, each time an infusion system is set up, a new infusion administration set may be utilized.

In some aspects, infusion system 102 further comprises a user interface coupled to or integrated in the infusion pump controller configured to display information related to the infusion system, and received inputs from a user for controlling the infusion system.

System 100 includes a hospital information system (HIS) 110 of the medical center in which the infusion system 102 is located. HIS 110 may include one or more computing devices, one or more databases, and the like. For example, HIS 110 is configured with an electronic medical recordation (EMR) system, a laboratory system, a pharmacy system, a medication ordering system, and the like, for the medical center, to facilitate communications such as transmitting and receiving laboratory testing data, prescription data, documentation of a patient's medical history, diagnosis, treatments, laboratory data, and/or the like.

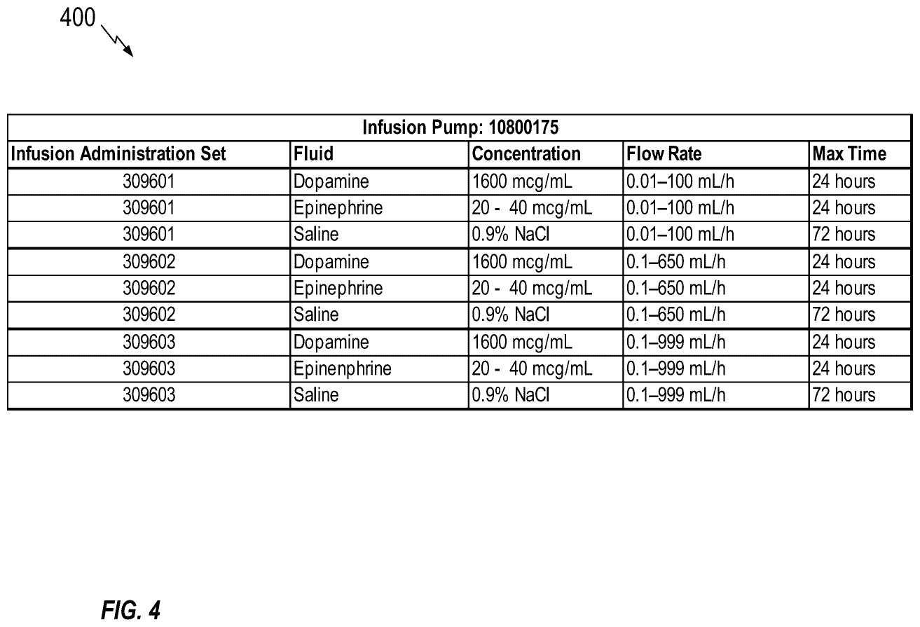

System 100 further includes an infusion compatibility component 106. The infusion compatibility component 106 is configured to detect compatibility of an infusion administration set coupled to the infusion system 102, and the infusion order. For example, the infusion compatibility component 106 may be configured to receive an identifier for the infusion administration set coupled to the infusion system 102 and determine information characterizing the infusion administration set. Information about the infusion order is also received, for example, an identifier of the fluid to be administered by the infusion system 102, and in some cases, other infusion information, such as operational parameters, environmental conditions, and the like of the infusion system 102. The infusion compatible component 106 is then configured to detect compatibility between the infusion administration set and the infusion order based on the information. In aspects, infusion compatibility component 106 is configured to utilize a compatibility table for the infusion system 102, e.g., stored in infusion set data store 108, to detect compatibility. An example compatibility table is depicted in FIG. 4. Then, if the infusion order and the infusion administration set is detected as compatible, the infusion system 102 may then commence infusion. If the infusion system 102 detects the infusion administration set is not compatible with the infusion order, the infusion system 102 may notify a user of the incompatibility. In some aspects, if the infusion system 102 detects the infusion administration set is not compatible with the infusion order, the infusion system 102 may prevent starting the infusion with the incompatible infusion administration set.

Aspects of system 100 may communicate (e.g., send and/or receive), directly or indirectly via one or more devices using network 104 (e.g., a local area network (LAN), internet, etc.).

Although depicted as separate components, in some aspects, components of system 100 may be integrated, for example, infusion compatibility component 106 may be integrated with the infusion system 102.

### Example Workflow for Compatibility Detection

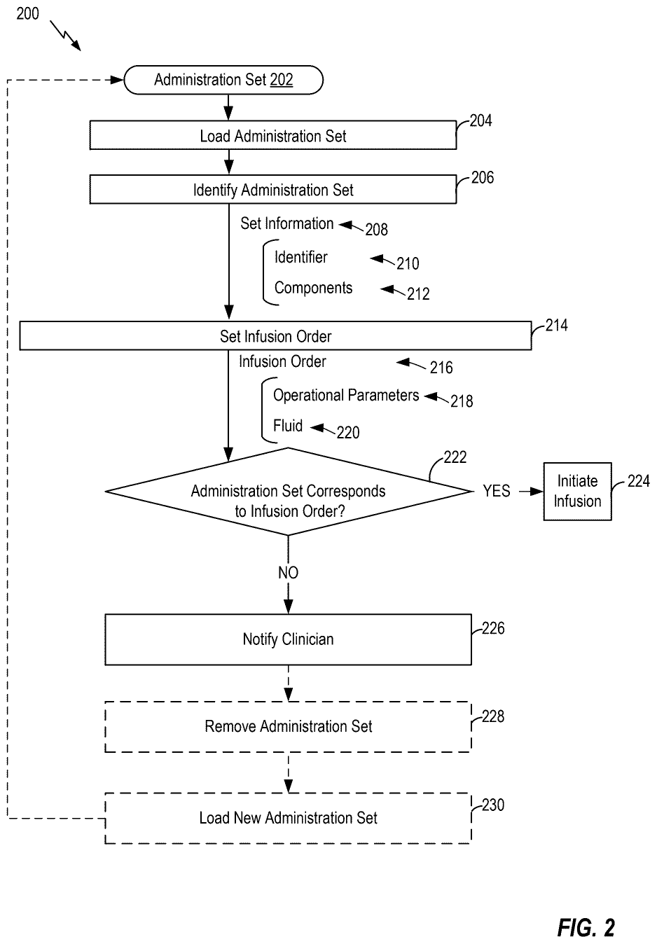

FIG. 2 depicts an example workflow 200 for detecting compatibility between an infusion administration set and infusion order, such as with infusion system 102 of system 100 in FIG. 1.

Initially, workflow 200 begins when an infusion administration set 202 is loaded into infusion system 102 at block 204. In some aspects, loading an infusion administration set 202 may comprise coupling one or more components of the infusion administration set 202 to the infusion system 102. In some implementations, the infusion system 102 may be configured to detect that the infusion administration set 202 is loaded. For example, the infusion system 102 may include a sensor to detect placement of tubing or a safety claim at a specific location of the infusion system 102.

At block 206, workflow 200 proceeds with identifying the infusion administration set 202. In some aspects, the infusion administration set 202 may be identified by an identifier 210 associated with the infusion administration set 202. For example, an identifier 210 of the infusion administration set 202 may be entered to a user interface of infusion system 102.

In some aspects, the identifier 210 may be detected on the infusion administration set 202. For example, an identifier may be a tag coupled to the infusion administration set 202 and scanned by a scanning device coupled to infusion system 102. In some examples, a tag may comprise a barcode, an RFID tag, a QR code, and the like, which may convey the identifier when scanned by the scanning device.

In some aspects, the identifier 210 may be determined based on measuring characteristics of the infusion administration set 202 loaded into the infusion system 102. For example, the infusion system 102 may use one or more infusion system sensors coupled to the infusion system 102 to measure one or more characteristics of the infusion administration set 202, and identify the infusion administration set 202 based on the one or more measured characteristic values. The infusion system 102 may be configured with known infusion administration set types, and physical characteristics of one or more components of an infusion administration set. Based on the measured characteristics, the infusion system 102 may identify the type of infusion administration set by comparing the measured characteristics and the known physical characteristics for one or more components of various types of infusion administration sets.

In some aspects, an identifier 210 of the infusion administration set 202 may indicate the type of the infusion administration set 202. The identifier 210 may be used to obtain information characterizing the infusion administration set 202 from an infusion administration data store, such as data store 108.

The information 208 characterizing the infusion administration set 202 may include component information 212 about one or more components of the infusion administration set 202. For example, a type of one or more components of the infusion administration set 202. The information characterizing the infusion administration set 202 may include a size of one or more of the components of the infusion administration set 202. The information characterizing the infusion administration set 202 may include a chemical property of the infusion administration set 202. For example, the infusion administration set may include one or more components comprised of non-DHP material, non-leaching material with respect to particular infusate, non-absorbing material with respect to particular infusate, ultraviolet (UV) resistant or blocking material, etc.

The information characterizing the infusion administration set 202 may include information contained in an instruction manual, an instruction for use document, a user manual, a manufacturer's specification document, and the like regarding the infusion administration set 202. The information characterizing the infusion administration set 202 may comprise an age of the infusion administration set 202, an age of one or more components of the infusion administration set 202. For example, the age of the infusion administration set 202 or one or more components of the infusion administration set 202, may be determined based on a manufacture date of the infusion administration set 202. In some cases, a manufacture date of the infusion administration set 202 may be based on a lot number, manufacturing batch, and the like of one or more components of the infusion administration set 202.

At block 214, workflow 200 proceeds with setting an infusion order 216 for the infusion system 102. In some aspects, the infusion order 216 includes a set of operational parameters 218 for operating the infusion system 102, and an identifier of a fluid 220 for infusion with infusion system 102.

In some aspects, a set of operational parameters 218 may include an infusion flow rate, a total volume to be infused, a total infusion time, a time of use of the infusion system 102, a total use time of the infusion system 102, and the like.

In some aspects, the infusion order 216 is received from a hospital information system, such as hospital information system 110, an electronic medical record, medication ordering system, a pharmacy system, and the like.

In some aspects, the infusion order 216 is received via a user interface of the infusion system 102. For example, an operational parameters control element may be displayed on the user interface of the infusion system 102, e.g., an electronic display associated with the infusion system. A user may select or input the one or more operational parameters via the user interface. In another example, a fluid selection control element may be displayed on the user interface of the infusion system 102, and a user may select or input the fluid identifier.

At block 222, workflow 200 detects compatibility of the infusion administration set 202 with the infusion order 216. Compatibility may be detected based on information characterizing the infusion administration set 202 and the infusion order 216. For example, in some aspects, the infusion administration set 202 may be determined to be compatible with the fluid 220 for the infusion order 216. In another example, the infusion administration set 202 may be determined to be compatible with the operational parameters 218 for the infusion order 216.

Compatibility may be determined based on a comparison between one or more characteristics of the infusion administration set 202 and one or more characteristics of the infusion order 216. For example, a compatibility table may list a set of fluids compatible with the infusion administration set 202. Where the fluid 220 is on the list of fluids compatible with the infusion administration set 202 in the compatibility table, the infusion administration set 202 may be detected as compatible for the fluid 220. As another example, the compatibility table may list a set of operational parameters compatible with the infusion administration set 202, for example, a range for each operational parameter of the set of operational parameters, a maximum threshold for each operational parameter, or a minimum threshold for each operational parameter. Where the set of operational parameters satisfies each operational parameter listed on the compatibility table for the infusion administration set 202, the infusion administration set 202 may be detected as compatible for the set of operational parameters 218.

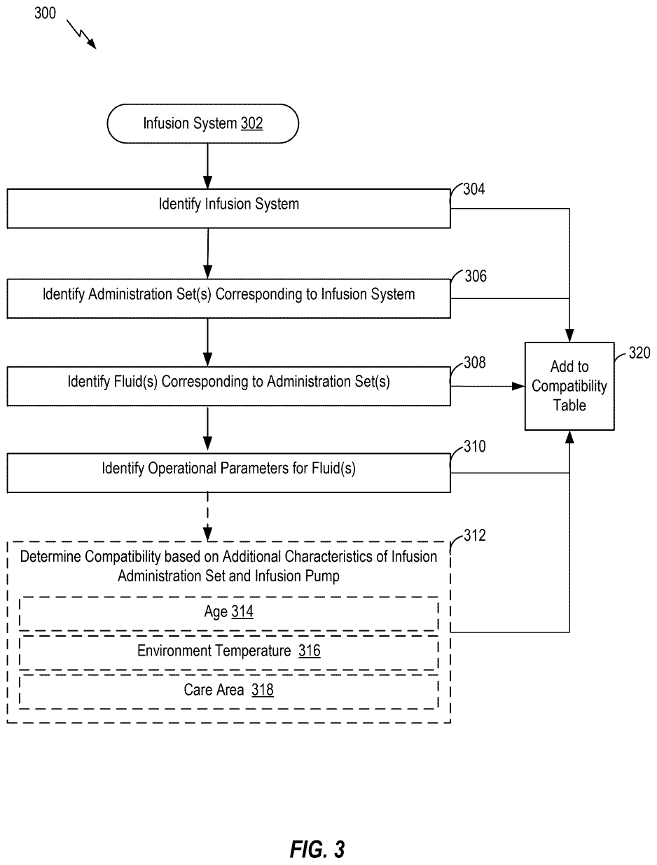

Additionally, in some aspects, the compatibility table may include additional characteristics of infusion administration sets, operational parameters, and fluids which may indicate compatibility. FIG. 3 describes generation of a compatibility table, and FIG. 4 depicts an example compatibility table.

For example, in some aspects, compatibility between the infusion administration set 202 and the infusion order 216 may be based on one or more characteristics of the infusion administration set 202, such as materials of the infusion administration set. For example, compatibility may be detected based on identifying an adverse interaction between a chemical property of a material of the infusion administration set and the fluid.

In another example, in some aspects, compatibility between the infusion administration set 202 and the infusion order 216 may be based on one or more characteristics of the fluid 220. For example, a characteristic of the fluid 220 may be a type of fluid, such as a medication, blood, saline, dextrose, minerals, and the like. The compatibility table may list which types of fluid are compatible with each infusion administration set. In another example, a characteristic of the fluid 220 may be a concentration of the fluid 220, such as a concentration above a threshold, below a threshold, out of a range, and the like. The compatibility table may list which concentrations of which types of fluids are compatible with each infusion administration set.

In some aspects, workflow 200 may further comprise obtaining one or more environmental conditions associated with the infusion system 102. For example, environmental conditions of the infusion system 102 may include an ambient temperature of the environment of the infusion system 102 (e.g., room temperature). An ambient temperature may be obtained by a thermometer associated with the infusion system 102, or based on a HVAC setting of the environment of the infusion system 102, obtained through a hospital information system.

As another example, environmental conditions of the infusion system 102 may include a care center location of the infusion system 102. Example care centers may include a neonatal intensive care unit (NICU), an intensive care unit (ICU), a pediatrics unit, oncology unit, medical-surgical unit, operating room, emergency department, outpatient clinic, critical care, post-anesthesia care unit (PACU), a patient's home, and other units or areas of patient care.

Environmental conditions may also include humidity levels, ultraviolet (UV) exposure, biodegradation, oxygen, heat, contaminants, and the like.

In some aspects, compatibility between the infusion administration set 202 and the infusion order 216 may be based on the environmental conditions associated with the infusion system 102. For example, a first infusion administration set may be compatible with a first infusion order for a first environmental temperature range. However, in the first infusion administration set with the first infusion order may not be compatible for a second environmental temperature. In some cases, a compatible environmental temperature may be part of an environmental temperature range, above an environmental temperature threshold, or below an environmental temperature threshold. In one example, the compatibility table may list an infusion administration set is compatible with an infusion order when the environmental temperature is within a given range.

In another example, a first infusion administration set may be compatible with a first infusion order in a particular care center or set of care centers, whereas the first infusion administration set may not be compatible with the first infusion order in a second care center or set of care centers. In one example, the compatibility table may list an infusion administration set is compatible with an infusion order when the location of the infusion system matches a set of compatible care centers.

In some aspects, compatibility between the infusion administration set 202 and the infusion order 216 may be based on one or more characteristics of a patient associated with the infusion pump. Characteristics of a patient may include, for example, a patient age, a patient weight, a patient criticality level, and the like. In one example, the compatibility table may list an infusion administration set is compatible with an infusion order when the patient's weight is within a patient weight range.

If, at block 222, the administration set 202 is detected as compatible with the infusion order 216, then workflow 200 proceeds to block 224 with initiating the infusion at infusion system 102. In some aspects, initiating the infusion at infusion system 102 may include activating an initiate infusion control element at the infusion system 102. For example, an initiate infusion control element may be displayed on a user interface associated with the infusion system 102. Upon selection of the initiate infusion control element, e.g., an input at the user interface, the infusion system 102 may initiate infusing the fluid through the infusion pump.

In some aspects, initiating the infusion at infusion system 102 may include commencing infusion of the fluid through the infusion pump of infusion system 102.

If, at block 222, the administration set 202 is detected as incompatible with the infusion order 216, then workflow 200 proceeds to block 226 with notifying a clinician, for example, at the infusion system 102. The notification may comprise an indication that the infusion administration set 202 is incompatible with the infusion order 216.

In some aspects, notifying at clinician at block 226 may comprise transmitting to an electronic medical record an indication of incompatibility. In some aspects, the notification may indicate the infusion administration set 202 is incompatible with the infusion order 216 due to an incompatibility with the fluid 220, or one or more operational parameters 218.

In some aspects, a notification to the clinician may be outputted on a user interface of infusion system 102.

In some aspects, the notification may be displayed as a popup, or other notification displayed on the user interface. A notification may be dismissed or overruled by a clinician.

In response to the notification, in some cases, workflow 200 proceeds to block 228 when the clinician may unload or uncouple the infusion administration set 202 from the infusion system 102. Then, at block 230, the clinician may load a new infusion administration set. Then, workflow 200 may repeat until a compatible infusion administration set is detected and the infusion may commence at block 224.

Note that workflow 200 is just one example, and other flows including fewer, additional, or alternative steps, consistent with this disclosure, are possible.

### Example Workflow for Generating a Compatibility Table

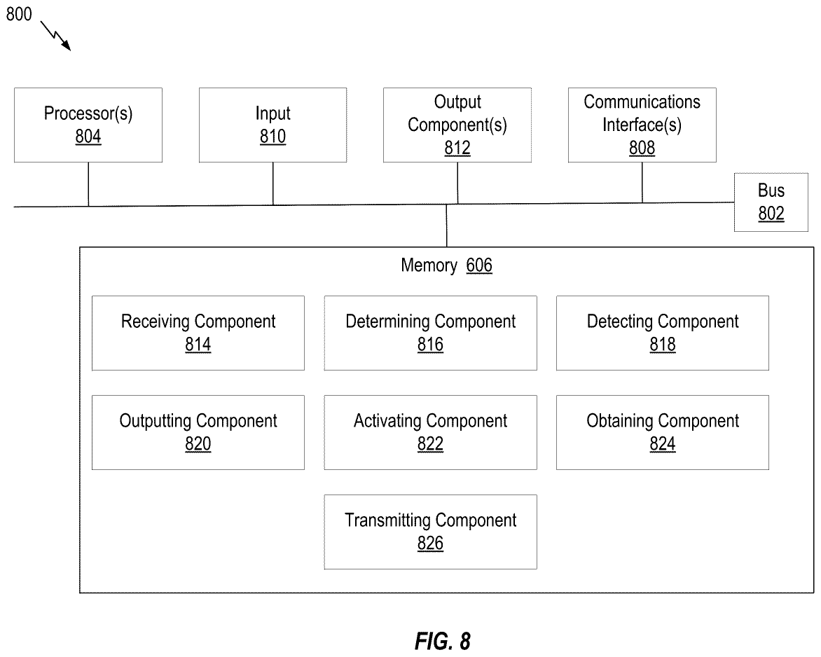

FIG. 3 depicts an example workflow 300 for generating a compatibility table, such as compatibility table 400 depicted in FIG. 4. In some aspects, compatibility table may be stored in data store 108 in system 100 of FIG. 1. The workflow 300 may be performed under control or direction of a computing device including one or more of the devices shown in FIG. 1, or computing device 800 in FIG. 8.

Initially, at block 304, an infusion system 302 may be identified. An infusion system 302 may be identified based on a type of infusion system.

A compatibility table for the infusion system may be generated at block 320. Example compatibility table 400 in FIG. 4, is generated for an infusion pump with identifier 10800175.

At block 306, a set of infusion administration sets configured for use with the infusion system may be identified. Infusion administration sets may be configured for use with an infusion system based on a manufacture's instruction, a physical connectivity, and the like. For example, one or more infusion administration sets may be configured for a particular type of infusion system.

In some aspects, the set of infusion administration sets configured for use with the infusion system may be further determined based on a set of available infusion administration sets. For example, an infusion system located in a hospital may be configured for a first set of infusion administration sets available in the hospital. Meanwhile, only a subset of infusion administration sets may be available for an infusion system in an outpatient clinic and the infusion system in the outpatient clinic is configured for compatibility with the subset of infusion administration sets.

The set of infusion administration sets identified as compatible with the infusion system may be added to a compatibility table for the infusion system at block 320.

In the compatibility table 400 in FIG. 4, three infusion administration sets are listed as compatible with infusion pump 10800175, first compatible set 309601, second compatible set 309602, and third compatible set 309603.

At block 308, a set of fluids compatible with each infusion administration set of the sets identified at block 306 may be identified. A fluid may be determined to be compatible with an infusion administration set based on characteristics of fluid and the infusion administration set, such as type and/or concentration.

Because different fluids may have different viscosities or other priorities, different infusion administration sets may be configured for different types of fluids. For example, a first infusion administration set may be configured for saline infusion, whereas a second infusion administration set may be configured for blood transfusion. The second infusion administration set may include a filter, which is configured to filter clots before a blood transfusion and thus is compatible with blood. Some infusion administration sets may include filters, connectors, locks, tubing, ports, regulators, or chambers which may be used for administration of different fluids. In some aspects, compatibility of an infusion administration set with a fluid may be determined based on a manufacture's instruction, a physical connectivity, and the like.

In some aspects, a fluid compatibility may be further determined based on a concentration of the fluid. For example, a first infusion administration set may be configured to administer a fluid at a first concentration, but not at a second concentration. Because the same fluid at different concentrations may have different viscosities or other properties, different infusion administration sets may be configured for different concentrations of the same fluid. A larger diameter tubing, a different kind of filter, or other components of an infusion administration set may make it compatibility for a higher concentration of fluid.

In some aspects, a fluid compatibility may be identified based on material of the infusion administration set, for example, based on a chemical property of the material of the infusion administration set. For example, compatibility may be detected based on identifying an adverse interaction between a chemical property of a material of the infusion administration set and the fluid.

The one or more types of fluids identified as compatible with each infusion administration set may be added to the compatibility table for each infusion administration set for the infusion system at block 320. In some aspects, one or more concentrations for each type of fluid identified as compatible may be added to the compatibility table.

In the compatibility table 400 in FIG. 4, for each of the three compatible infusion administration sets, three different compatible fluids are listed. In the depicted example, first compatible set 309601 is listed as compatible with dopamine, epinephrine, and saline. Additionally, compatible concentrations or ranges are listed for each fluid.

At block 310, a set of operational parameters for each fluid listed as compatible for each infusion administration set identified at block 308 is identified. Operational parameters may include an infusion flow rate, a total volume to be infused, a total infusion time, a time of use of the infusion system, a total use time of the infusion system, and the like. Some infusion administration sets may be limited to safe operation within certain operational ranges, or above or below certain operational thresholds. For example, a flow rate above an operational threshold may be too high for an infusion administration set due to the pressure to maintain the flow rate. As another example, an infusion administration set may be configured for 24 hours of fluid administration due to the materials of the infusion administration set.

In some aspects, compatible operational parameter values may comprise a range of values, a maximum threshold, or a minimum threshold. An operational parameter may be detected as compatible (such as at block 222 of FIG. 2), where the operational parameter is within the range, below a maximum threshold, or above a minimum threshold. Each operational parameter range or threshold identified as compatible with each type of fluid (and in some cases concentration), may be added to the compatibility table for each infusion administration set for the infusion system.

The one or more sets of operational parameters identified as compatible for each infusion administration set may be added to the compatibility table at block 320.

In the compatibility table 400 in FIG. 4, for each of the three compatible infusion administration sets, additional operational parameters are listed. In this example, first compatible set 309601 is listed as compatible with fluids at a flow rate between 0.01 mL/h and 100 mL/h, whereas second compatible set 309602 is compatible with fluids at a flow rate between 0.1 mL/h and 650 mL/h. The third compatible set 309603 is compatible with fluids at a flow rate between 0.1 mL/h and 999 mL/h. Additionally, a max user time is listed for each fluid. For example, for all infusion administration sets where the fluid is dopamine or epinephrine, the infusion administration set has a max use time of 24 hours, whereas for saline, the max use time is 72 hours.

In some aspects, a compatibility table may be further configured based on additional characteristics of the infusion administration set. For example, in some aspects, workflow 300 may proceed with block 312 with determining compatibility based on one or more characteristics of the infusion administration set and/or the infusion system.

For example, one characteristic may be an age of the infusion administration set, such as based on a manufacturing date. An older infusion administration set may not be able to handle higher flow rate, more viscous fluid, or a longer time of use compared to a new infusion administration set, and thus may have a narrower compatibility. In some aspects, at block 314, compatible age(s) of infusion administration sets may be determined and then, at block 320, added to the compatibility table.

In some aspects, a compatibility table may be further configured based on an environmental temperature of the infusion system. An infusion administration set may be configured for use within an environmental temperature range, above a certain temperature, or below a certain temperature. In some aspects, at block 316, compatible environmental temperature(s), e.g., a range of compatible temperature, of infusion system may be determined and then, at block 320, added to the compatibility table.

In some aspects, a compatibility table may be further configured based on a care center of the infusion system. An infusion administration set may be configured for a pediatric or neonate care center because the size of the infusion administration set may be small for use in pediatric or neonate patients. An infusion administration set may be configured for adult care centers because the size of the infusion administration set may be too large for use in pediatric or neonate patients. In some aspects, at block 318, compatible care area(s) of infusion system may be determined and then, at block 320, added to the compatibility table.

In some aspects, a compatibility table may be generated for each infusion system based on one or more infusion administration sets, one or more fluids, and one or more sets of operational parameters. In some cases, a compatibility table may be further generated based on characteristics of the infusion administration set, environment of the infusion system, or characteristics of the infusion system.

The additional characteristics identified as compatible for each infusion administration set and infusion pump may be added to the compatibility table at block 320.

Note that workflow 300 is just one example, and other flows including fewer, additional, or alternative steps, consistent with this disclosure, are possible.

### Example Infusion System

FIG. 5 depicts an example infusion system 500, which may implement methods described herein to detect incompatible infusion administration sets. The infusion system 500 may be coupled to a patient 550 to administer a substance to the patient 550 through infusion. The infusion system 500 may include one or more pumps, for example, pump 502, pump 504, pump 506, and pump 508. Although a large volume pump is illustrated, other types of pumps may be implemented, such as a peristaltic pump, a small volume pump, a syringe pump, an anesthesia delivery pump, or a patient-controlled analgesic. A pump may be an infusion device configured to deliver a substance, (e.g., fluid, nutrients, drug, etc.) to a patient's circulatory system, or epidural space, for example, via an intravenous infusion, subcutaneous infusion, arterial infusion, epidural infusion, etc., or to a patient's digestive system, for example, via a nasogastric tube (NG), a percutaneous endoscopic gastrostomy tube (PEG), nasojejunal tube (NJ), etc. Each of the pumps 502, 504, 506, or 508 may comprise a standard infusion pumping unit, patient controlled analgesia (PCA) pump, syringe pump, pulse oximeter, invasive or non-invasive blood pressure monitor, electrocardiograph, bar code reader, printer, temperature monitor, RF telemetry link, fluid warmer/IV pump, or high rate IV pump (2000+ ml/hr).

Each of the pumps 502, 504, 506, or 508 may be fluidly connected with an upstream fluid line 512, fluid line 514, fluid line 516, and fluid line 518, respectively. Further, each of pump 502, pump 504, pump 506, and pump 508, may be fluidly connected with a downstream fluid line 522, fluid line 524, fluid line 526, and fluid line 528, respectively. The fluid lines may be any type of fluid conduit, such as tubing, through which fluid can flow.

Each of fluid supply 532, fluid supply 534, fluid supply 536, and fluid supply 538, may be a reservoir, for example, as bottles shown, are inverted and suspended above the pumps. Fluid supplies may also take the form of bags, syringes, or other types of contains. The medical device system 500 may be mounted on a roller stand or intravenous pole 540.

Infusion pump system 500 may further comprise check valves, drip chambers, valved ports, connectors, and other devices configured to administer a substance.

Each of the pumps 502, 504, 506, or 508 of infusion pump system 500 may be coupled to an infusion pump controller 560. The infusion pump controller 560 may be a patient care device (PCD) or patient care unit (PCU). The infusion pump controller 560 is configured to control operations of the pumps 502, 504, 506, or 508, for example, to control infusions to patient 550. the control of fluid delivery to the patient and the monitoring of the fluid path for occlusion or air-in-line

The infusion pump controller 560 may further include a user interface, for example, an information display such as a liquid crystal display, and/or a touch-screen. The user interface of the infusion pump controller 560 may be used during set up and/or operating procedures to facilitate control of the infusion system 500. For example, entry, editing, and/or display of operational parameters of the pumps 502, 504, 506, or 508. The infusion pump controller 560 may further include one or more softkeys and/or hardkeys to facilitate data entry and commands. In some aspects, a user interface may display the operational parameters of the pumps 502, 504, 506, or 508, for example, the infusion rate at which the pump is operating. The user interface may additionally and/or alternatively, be used to display informational, advisory, alarm, or malfunction messages associated with the pumps 502, 504, 506, or 508.

The infusion pump controller 560 may further be configured to provide power to the infusion system 500 and interface between aspects of the infusion system 500 and external devices and/or networks, for example, patient monitoring networks, instructional systems (e.g., an electronic medical record system), pharmacy systems, and/or nurse call systems, or as an interface to external equipment such as barcode readers to provide a means of inputting drug and/or patient information from medication or patient records. In some aspects, the infusion pump controller 560 is configured to provide physical attachment of the infusion system 500 to structures, such as intravenous pole 540 and/or bedrails, and the like.

### Example Syringe Pump

FIG. 6 depicts an example syringe pump 602. Syringe pump 602 includes a graphical user interface (GUI) 624 for displaying operational information, including the selected syringe type and the configured infusion (e.g., in terms of rate of infusion, volume infused, length of time of infusion, etc.). A user may operate syringe pump 602 through one or more buttons 622 in this example. Note that other examples may include other arrangements, such as touch-screen interfaces, or interfaces operable by remote device, such as by an application on a computing device.

Syringe 601 is shown next to syringe pump 602, rather than loaded in the pump, for clarity of illustration. Syringe pump 602 includes cradle 604 in which a barrel 603 of syringe 601 rests when mounted in the syringe pump 602. Sensor 628 detects volume markings along a barrel of syringe 601. Cradle 604 has a clamp 606 to securely hold the barrel 603 in a fixed position in cradle 604 to resist axial and lateral movement. Clamp 606 may pivot between an open position to permit loading or removal of syringe 601 and a closed position over cradle 604. Barrel clamp 606 may measure an outside diameter of the barrel 603 of syringe 601. Barrel flange 605 of syringe 601 resides in a barrel flange groove 608 in syringe pump 602 to immobilize barrel 603 from axial movement during movement of plunger 607 within barrel 603 of syringe 601. Barrel flange groove may measure an outside diameter and a thickness of the barrel flange 605 of syringe 601.

Plunger 607 can include push-button 609 having an inner side 611 and being interconnected with stopper 603 of plunger 607 by piston 615. When mounted in syringe pump 602, push-button 609 can be held by drive head 610 with a plunger retainer comprising a pair of pivotally mounted claws, first retainer claw 612 and second retainer claw 614, shown in the closed position in FIG. 6. The retainer claws 612 and 614 can curve inwardly toward each other to grasp push-button 609 mounted in syringe pump 602.

A rotation knob 616 can be used to control the positions of the first and second retainer claws 612 and 614 to allow removal and insertion of the push-button 609 and to release the split-nut from the driveshaft to permit axial positioning of the drive head 610. Syringes can be provided for use with a syringe pump with different quantities of fluid, and the plunger can be located at different positions in relation to the barrel.

The drive head 610 can allow manual adjustment to accommodate syringes with different beginning plunger positions. A syringe inserted in the cradle 604 can align with the drive head 610 within a particular axial range. The points where the axial center lines of the syringes intersect the driver can change according to the size of the syringe but only in one direction along the drive head 610. A guide device 618 can extend from the drive head 610 to a point within a body of the syringe pump 602.

Syringe pump 602 can include a control panel 620 providing multiple buttons 622 for control of the syringe pump 602 as well as GUI 624 used to present pump-specific information to the operator. The buttons 622 can allow the operator to program the syringe pump 602 for the flow rate, the volume to be infused, and other pump parameters. GUI 624 can present the programmed flow rate, the amount of fluid remaining to be infused, as well as alarms and other information.

The drive head 610 can include a contact plate 626 that has a pushing surface that contacts the outer side 617 of the push-button 609 as the drive head 610 moves forward toward the barrel 603, pushing the plunger 607 into the barrel 603 of the syringe to expel the syringe contents through a fluid administration set tubing 619 to a patient. A drive mechanism 654 is configured to drive the drive head 610 forward. The drive mechanism 654 may include, a drivetrain, for example, gears, axles, shafts, chains, hydraulics, and/or any other components for translating rotational motion of a motor into linear motion of the drive head 610. The drivetrain may include linear actuating components, such as a linear stepper motor, a brushed DC electric motor, a brushless DC electric motor, a servo motor, an AC motor, or any other type of motor.

When the contact plate 626 exerts force against the push-button 609, the force may be detected by force sensor 629 and transmitted to a processor for monitoring. In the case of friction between the stopper 613 and the barrel 603, the force exerted can increase and can be detected by force sensor 629.

Syringe pump 602 is configured with known syringe types containing information such as syringe inner diameter and stroke of the stopper 613. Syringe pump 602 determines the position of stopper 613 based on the movement of drive head 610 based on the syringe type and stored characteristics of the syringe type. Then, syringe pump 602 calculates the volume infused, time elapsed, volume remaining, and time remaining. As the drive head 610 continues to move, a flow rate is determined based on the characteristics of the syringe and the velocity of the drive head 610.

One or more sensors, such as sensor 628, barrel clamp 606, groove 608, claws 612, and 614, and force sensor 629, may measure various characteristics of syringe 601, to obtain a measured value for the characteristic, such as barrel length, barrel outside diameter, flange shape, flange size, plunger shape, plunger size (e.g., diameter), plunger tab thickness, volume per plunger distance moved, driver head height, etc. These sensors may include one or more of an optical sensor, a light source, a pressure sensor, a position sensor (e.g., magnetic linear position sensor indicating the plunger head), force sensor, and the like.

### Example Method for Detecting Infusion Administration Set Compatibility

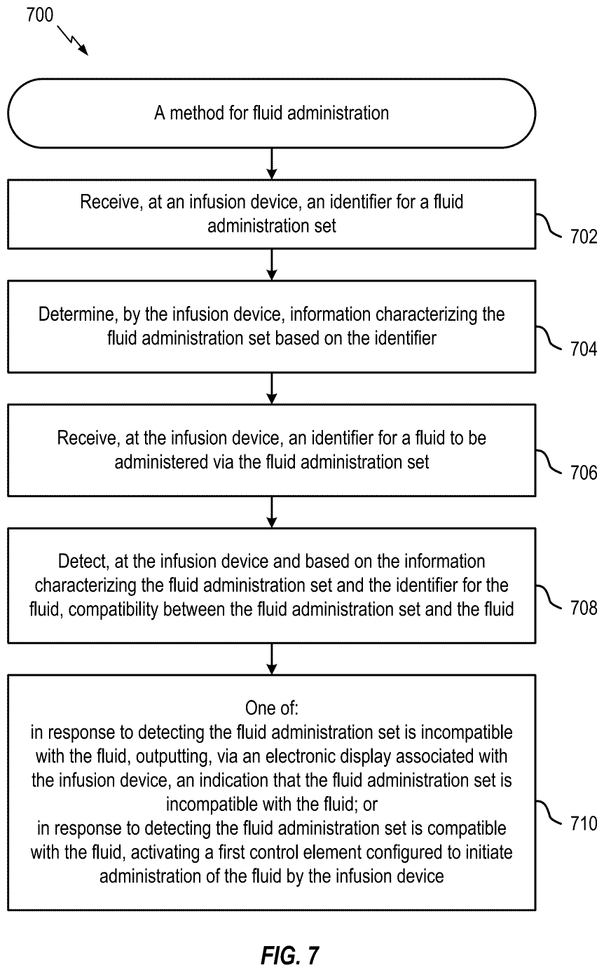

FIG. 7 shows an example method 700 for detecting infusion administration set compatibility by an infusion system, such as infusion system 500 in FIG. 5.

Method 700 begins at step 702 with receiving, at an infusion device, an identifier for a fluid administration set.

Method 700 then proceeds to step 704 with determining, by the infusion device, information characterizing the fluid administration set based on the identifier.

Method 700 then proceeds to step 706 with receiving, at the infusion device, an identifier for a fluid to be administered via the fluid administration set.

Method 700 then proceeds to step 708 with detecting, at the infusion device and based on the information characterizing the fluid administration set and the identifier for the fluid, compatibility between the fluid administration set and the fluid.

Method 700 then proceeds to step 710 with one of: in response to detecting the fluid administration set is incompatible with the fluid, outputting, via an electronic display associated with the infusion device, an indication that the fluid administration set is incompatible with the fluid; or in response to detecting the fluid administration set is compatible with the fluid, activating a first control element configured to initiate administration of the fluid by the infusion device

In some aspects, the compatibility between the fluid administration set and the fluid is further detected based on one or more characteristics of the fluid.

In some aspects, the one or more characteristics of the fluid comprise a type of the fluid.

In some aspects, the one or more characteristics of the fluid comprise a concentration of the fluid.

In some aspects, the information characterizing the fluid administration set comprises a type of one or more devices of the fluid administration set.

In some aspects, the information characterizing the fluid administration set comprises a size of one or more devices of the fluid administration set.

In some aspects, the information characterizing the fluid administration set comprises an age of one or more devices of the fluid administration set.

In some aspects, method 700 further includes receiving one or more operational parameters configured to control operation of a medical device for administration of the fluid, wherein the compatibility between the fluid administration set and the fluid is further detected based on the one or more operational parameters.

In some aspects, the medical device comprises an infusion system; and the one or more operational parameters comprise an infusion flow rate.

In some aspects, the medical device comprises an infusion system; and the one or more operational parameters comprise a total volume to be infused.

In some aspects, the medical device comprises an infusion system; and the one or more operational parameters comprise a total infusion time.

In some aspects, the one or more operational parameters are received from an electronic medical record.

In some aspects, method 700 further includes activating a second control element to select the one or more operational parameters, wherein the identifier is received in response to a selection of the second control element.

In some aspects, method 700 further includes obtaining one or more environmental conditions associated with a medical device for administration of the fluid, configured to control operation of a medical device for administration of the fluid, wherein the compatibility between the fluid administration set and the fluid is further determined based on the one or more environmental conditions.

In some aspects, the one or more environmental conditions comprises an ambient temperature of an environment of the medical device.

In some aspects, the one or more environmental conditions comprises a care center location of the medical device.

In some aspects, method 700 further includes activating a third control element to select the fluid to be administered via the fluid administration set, wherein the identifier is received in response to a selection of the third control element.

In some aspects, the identifier for the fluid to be administered via the fluid administration set is received from an electronic medical record.

In some aspects, method 700 further includes detecting the identifier of the fluid administration set on the fluid administration set.

In some aspects, method 700 further includes obtaining a set of measured characteristic values for one or more devices of the fluid administration set loaded in an infusion system from one or more infusion system sensors.

In some aspects, method 700 further includes determining the identifier of the fluid administration set based on the set of measured characteristic values.

In some aspects, method 700 further includes transmitting to an electronic medical record an indication of the compatibility in response to determining the fluid administration set is incompatible with the fluid.

In some aspects, the information characterizing the administration set comprises a chemical property of the administration set, and wherein detecting compatibility includes identifying an adverse interaction between the chemical property and the fluid.

In some aspects, the information characterizing the administration set comprises an instruction for use document, and wherein detecting compatibility includes determining whether the fluid is identified in the instruction for use document.

In one aspect, method 700, or any aspect related to it, may be performed by an apparatus, such as computing device 800 of FIG. 8, which includes various components operable, configured, or adapted to perform the method 700. Computing device 800 is described below in further detail.

Note that FIG. 7 is just one example of a method, and other methods including fewer, additional, or alternative steps are possible consistent with this disclosure.

### Example Computing Device for Infusion Administration Set Compatibility

FIG. 8 depicts an example computing device 800, such as a medical device, which implements various features and processes described herein, such as infusion system 102 in FIG. 1. For example, the computing device 800 may perform one or more steps of method 700. The computing device 800 may include one or more processors 804, one or more memories 808, one or more input components 810, one or more output components 812, and one or more communication interfaces 808. Each of these components may be coupled by a bus 802.

Computing device 800 may perform these processes based on one or more processors 804 executing software instructions stored by a computer-readable medium, such as one or more memories 808. In certain embodiments, one or more processors 804 may be programmed/designed/configured to perform these processes. A computer-readable medium (e.g., a non-transitory computer-readable medium) is defined herein as a non-transitory memory device. A memory device includes memory space located inside of a single physical storage device or memory space spread across multiple physical storage devices. Software instructions may be read into one or more memories from another computer-readable medium or from another device via communication interface 808. When executed, software instructions stored in one or more memories may cause one or more processors 804 to perform one or more processes described herein.

A memory 808 may include data storage or one or more data structures (e.g., a database, etc.). Computing device 800 may be capable of receiving information from, storing information in, communicating information to, or searching information stored in the data storage or one or more data structures in one or more memories 808.

A memory 808 may include random access memory (RAM), read only memory (ROM), and/or other types of dynamic or static storage devices (e.g., flash memory, magnetic memory, optical memory, etc.), that stores information and/or instructions for use by one or more processors 804. For example, a memory 808 may include different forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

In some aspects, memory 808 may include receiving component 814, determining component 816, detecting component 818, outputting component 820, activating component 822, obtaining component 824, and transmitting component 826.

For example, in some aspects, one or more memories 808 may include receiving component 814 configured for receiving, at an infusion device, an identifier for a fluid administration set. In some aspects, one or more memories 808 may include determining component 816 configured for determining, by the infusion device, information characterizing the fluid administration set based on the identifier. In some aspects, one or more memories 808 may include receiving component 814 configured for receiving, at the infusion device, an identifier for a fluid to be administered via the fluid administration set.

In some aspects, one or more memories 808 may include detecting component 816 configured for detecting, at the infusion device and based on the information characterizing the fluid administration set and the identifier for the fluid, compatibility between the fluid administration set and the fluid.

In some aspects, one or more memories 808 may include outputting component 820 configured for, in response to detecting the fluid administration set is incompatible with the fluid, outputting, via an electronic display associated with the infusion device, an indication that the fluid administration set is incompatible with the fluid.

In some aspects, one or more memories 808 may include activating component 822 configured for, in response to detecting the fluid administration set is compatible with the fluid, activating a first control element configured to initiate administration of the fluid by the infusion device.

In some aspects, one or more memories 808 may include obtaining component 824 configured for, obtaining data. For example, obtaining component 824 may obtain one or more environmental conditions associated with the infusion device for administration of the fluid, configured to control operation of the infusion device for administration of the fluid, wherein the compatibility between the fluid administration set and the fluid is further determined based on the one or more environmental conditions. In another example, obtaining component 824 may obtain a set of measured characteristic values for one or more devices of the fluid administration set loaded in an infusion system from one or more infusion system sensors; and determining the identifier of the fluid administration set based on the set of measured characteristic values.

In some aspects, one or more memories 808 may include transmitting component 826, configured for, transmitting data. For example, transmitting component 826 may be configured to transmit to an electronic medical record an indication of the compatibility in response to determining the fluid administration set is incompatible with the fluid.

One or more processors 804 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.), that may be programmed to perform a function, such as described herein.

One or more input components 810 may include a component that permits computing device 800 to receive information, such as via user input (e.g., a touch screen display, a keyboard, a keypad, a mouse, a button, a switch, a microphone, etc.). Further, one or more input components 810 may include a sensor for sensing information (e.g., a global positioning system (GPS) component, an accelerometer, a gyroscope, an actuator, etc.).

One or more output components 812 may include a component that provides output information from computing device 800 (e.g., a display, a speaker, one or more light-emitting diodes (LEDs), etc.).

Communication interface 808 may include a transceiver-like component (e.g., a transceiver, a separate receiver and transmitter, etc.) that enables computing device 800 to communicate with other devices, such as via a wired connection, a wireless connection, or a combination of wired and wireless connections. Communication interface 808 may permit computing device 800 to receive information from another device and/or provide information to another device. For example, communication interface 808 may include an Ethernet interface, an optical interface, a coaxial interface, an infrared interface, a radio frequency (RF) interface, a universal serial bus (USB) interface, a Wi-Fi interface, a cellular network interface, and/or the like.

### Example Clauses

Implementation examples are described in the following numbered clauses:

Clause 1: A method, comprising: receiving, at an infusion device, an identifier for a fluid administration set; determining, by the infusion device, information characterizing the fluid administration set based on the identifier; receiving, at the infusion device, an identifier for a fluid to be administered via the fluid administration set; detecting, at the infusion device and based on the information characterizing the fluid administration set and the identifier for the fluid, compatibility between the fluid administration set and the fluid; and one of: in response to detecting the fluid administration set is incompatible with the fluid, outputting, via an electronic display associated with the infusion device, an indication that the fluid administration set is incompatible with the fluid; or in response to detecting the fluid administration set is compatible with the fluid, activating a first control element configured to initiate administration of the fluid by the infusion device.

Clause 2: The method of Clause 1, wherein the compatibility between the fluid administration set and the fluid is further detected based on one or more characteristics of the fluid.

Clause 3: The method of Clause 2, wherein the one or more characteristics of the fluid comprise a type of the fluid.

Clause 4: The method of Clause 2, wherein the one or more characteristics of the fluid comprise a concentration of the fluid.

Clause 7: The method of any one of Clauses 1-4, wherein the information characterizing the fluid administration set comprises a type of one or more devices of the fluid administration set.

Clause 8: The method of any one of Clauses 1-7, wherein the information characterizing the fluid administration set comprises a size of one or more devices of the fluid administration set.

Clause 7: The method of any one of Clauses 1-8, wherein the information characterizing the fluid administration set comprises an age of one or more devices of the fluid administration set.

Clause 8: The method of any one of Clauses 1-7, further comprising: receiving one or more operational parameters configured to control operation of the infusion pump for administration of the fluid, wherein the compatibility between the fluid administration set and the fluid is further detected based on the one or more operational parameters.

Clause 9: The method of Clause 8, wherein the one or more operational parameters comprise an infusion flow rate.

Clause 10: The method of Clause 8, wherein the one or more operational parameters comprise a total volume to be infused.

Clause 11: The method of Clause 8, wherein the one or more operational parameters comprise a total infusion time.

Clause 12: The method of Clause 8, wherein the one or more operational parameters are received from an electronic medical record.

Clause 13: The method of Clause 8, further comprising: activating a second control element to select the one or more operational parameters, wherein the identifier is received in response to a selection of the second control element.

Clause 14: The method of any one of Clauses 1-13, further comprising: obtaining one or more environmental conditions associated with the infusion device for administration of the fluid, configured to control operation of the infusion device for administration of the fluid, wherein the compatibility between the fluid administration set and the fluid is further determined based on the one or more environmental conditions.

Clause 15: The method of Clause 14, wherein the one or more environmental conditions comprises an ambient temperature of an environment of the infusion device.

Clause 16: The method of Clause 14, wherein the one or more environmental conditions comprises a care center location of the infusion device.

Clause 17: The method of any one of Clauses 1-18, further comprising: activating a third control element to select the fluid to be administered via the fluid administration set, wherein the identifier is received in response to a selection of the third control element.

Clause 18: The method of any one of Clauses 1-17, wherein the identifier for the fluid to be administered via the fluid administration set is received from an electronic medical record.

Clause 19: The method of any one of Clauses 1-18, further comprising detecting the identifier of the fluid administration set on the fluid administration set.

Clause 20: The method of any one of Clauses 1-19, further comprising: obtaining a set of measured characteristic values for one or more devices of the fluid administration set loaded in an infusion system from one or more infusion system sensors; and determining the identifier of the fluid administration set based on the set of measured characteristic values.

Clause 21: The method of any one of Clauses 1-20, further comprising transmitting to an electronic medical record an indication of the compatibility in response to determining the fluid administration set is incompatible with the fluid.

Clause 22: The method of any one of Clauses 1-21, wherein the information characterizing the administration set comprises a chemical property of the administration set, and wherein detecting compatibility includes identifying an adverse interaction between the chemical property and the fluid.

Clause 23: The method of any one of Clauses 1-22, wherein the information characterizing the administration set comprises an instruction for use document, and wherein detecting compatibility includes determining whether the fluid is identified in the instruction for use document.

Clause 24: An infusion system, comprising: an infusion pump configured to deliver fluid to a patient; and an infusion pump controller configured to perform a method in accordance with any one of Clauses 1-23.

Clause 27: A processing system, comprising: memory comprising computer-executable instructions; and one or more processors configured to execute the computer-executable instructions and cause the processing system to perform a method in accordance with any one of Clauses 1-23.

Clause 28: A processing system, comprising means for performing a method in accordance with any one of Clauses 1-23.

Clause 27: A non-transitory computer-readable medium storing program code for causing a processing system to perform the steps of any one of Clauses 1-23.

Clause 28: A computer program product embodied on a computer-readable storage medium comprising code for performing a method in accordance with any one of Clauses 1-23.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements, and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some” refers to one or more.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An infusion system, comprising:
an infusion pump configured to deliver fluid to a patient; and
an infusion pump controller configured to:
receive, at the infusion pump controller, an identifier for a fluid administration set;
determine, by the infusion pump controller, information characterizing the fluid administration set based on the identifier;
receive, at the infusion pump controller, an identifier for a fluid to be administered via the fluid administration set;
detect, at the infusion pump controller and based on the information characterizing the fluid administration set and the identifier for the fluid, compatibility between the fluid administration set and the fluid; and
one of:
in response to detecting the fluid administration set is incompatible with the fluid, output, via an electronic display associated with the infusion pump controller, an indication that the fluid administration set is incompatible with the fluid; or
in response to detecting the fluid administration set is compatible with the fluid, activate a first control element configured to initiate administration of the fluid by the infusion pump.

2. The infusion system of claim 1, wherein the compatibility between the fluid administration set and the fluid is further determined based on one or more characteristics of the fluid.

3. The infusion system of claim 2, wherein the one or more characteristics of the fluid comprise a type of the fluid.

4. The infusion system of claim 2, wherein the one or more characteristics of the fluid comprise a concentration of the fluid.

5. The infusion system of claim 1, wherein the information characterizing the fluid administration set comprises a type of one or more devices of the fluid administration set.

6. The infusion system of claim 1, wherein the information characterizing the fluid administration set comprises a size of one or more devices of the fluid administration set.

7. The infusion system of claim 1, wherein the information characterizing the fluid administration set comprises an age of one or more devices of the fluid administration set.

8. The infusion system of claim 1, wherein the infusion pump controller is further configured to receive one or more operational parameters configured to control operation of an infusion device for administration of the fluid, wherein the compatibility between the fluid administration set and the fluid is further determined based on the one or more operational parameters.

9. The infusion system of claim 8, wherein the one or more operational parameters are received from an electronic medical record.

10. The infusion system of claim 8, wherein the infusion pump controller is further configured to:
activate a second control element to select the one or more operational parameters, wherein the identifier is received in response to a selection of the second control element.

11. The infusion system of claim 1, wherein the infusion pump controller is further configured to obtain one or more environmental conditions associated with an infusion device for administration of the fluid, configured to control operation of a infusion device for administration of the fluid, wherein the compatibility between the fluid administration set and the fluid is further determined based on the one or more environmental conditions.

12. The infusion system of claim 11, wherein the one or more environmental conditions comprises at least one of an ambient temperature of an environment of the infusion system or a care center location of the infusion system.

13. The infusion system of claim 1, wherein the infusion pump controller is further configured to activate a third control element to select the fluid to be administered via the fluid administration set, wherein the identifier is received in response to a selection of the third control element.

14. The infusion system of claim 1, wherein the identifier for the fluid to be administered via the fluid administration set is received from an electronic medical record.

15. The infusion system of claim 1, wherein the infusion pump controller is further configured to detect the identifier of the fluid administration set on the fluid administration set.

16. The infusion system of claim 1, wherein the infusion pump controller is further configured to:
obtain a set of measured characteristic values for one or more devices of the fluid administration set loaded in an infusion system from one or more infusion system sensors; and
determine the identifier of the fluid administration set based on the set of measured characteristic values.

17. The infusion system of claim 1, wherein in response to determining the fluid administration set is incompatible with the fluid, the infusion pump controller is further configured to transmit to an electronic medical record an indication of the compatibility.

18. The infusion system of claim 1, wherein the information characterizing the administration set comprises a chemical property of the administration set, and wherein detecting compatibility includes identifying an adverse interaction between the chemical property and the fluid.

19. The infusion system of claim 1, wherein the information characterizing the administration set comprises an instruction for use document, and wherein detecting compatibility includes determining whether the fluid is identified in the instruction for use document.

20. A method, comprising:
receiving, at an infusion device, an identifier for a fluid administration set;
determining, by the infusion device, information characterizing the fluid administration set based on the identifier;
receiving, at the infusion device, an identifier for a fluid to be administered via the fluid administration set;
detecting, at the infusion device and based on the information characterizing the fluid administration set and the identifier for the fluid, compatibility between the fluid administration set and the fluid; and
one of:
in response to detecting the fluid administration set is incompatible with the fluid, outputting, via an electronic display associated with the infusion device, an indication that the fluid administration set is incompatible with the fluid; or
in response to detecting the fluid administration set is compatible with the fluid, activating a first control element configured to initiate administration of the fluid by the infusion device.
